(19) United States
(12) Patent Application Publication
(10) Pub. No.: US 2023/0273217 A1
(43) Pub. Date: Aug. 31, 2023

(54) DERIVATIZATION OF AT LEAST ONE ANALYTE OF INTEREST FOR MASS SPEC MEASUREMENTS IN PATIENT SAMPLES

(71) Applicant: Roche Diagnostics Operations, Inc., Indianapolis, IN (US)

(72) Inventors: Kathrin Arnhard, Landsberg am Lech (DE); Christian Clemens, Dannstadt-Schauernheim (DE); Gaston Hubertus Maria Vondenhoff, Muenchen (DE)

(21) Appl. No.: 18/312,442

(22) Filed: May 4, 2023

Related U.S. Application Data
(63) Continuation of application No. PCT/EP2021/079748, filed on Oct. 27, 2021.

(30) Foreign Application Priority Data
Nov. 5, 2020 (EP) 20206044.8

(51) Int. Cl. G01N 33/68; G01N 33/543; G01N 33/94; G01N 30/72

(52) U.S. Cl. CPC G01N 33/6848; G01N 33/54326; G01N 33/9473; G01N 33/9486; G01N 30/72

(57) ABSTRACT

The present invention relates to a method for determining the presence or level of an analyte of interest and the use thereof. Further, present invention relates to an analytical system, a sampling tube and the use of the sampling tube and a nucleophilic derivatization reagent.

### FIELD OF THE INVENTION

The present invention relates to a method for determining the presence or level of an analyte of interest and the use thereof. Further, the present invention relates to an analytical system, a sampling tube and the use of the sampling tube and a nucleophilic derivatization reagent.

### BACKGROUND OF THE INVENTION

Analytes of interest can be small molecules, e.g. valproic acid or salicylic acid, having a molar mass mass of smaller than 200 Da. It is difficult to fragment these small molecules by mass spectrometry, because they do not show any characteristic fragmentation patterns.

Valproic acid (1, e.g. scheme 2) is a small molecule with a molecular weight of only 144 g/mol, which is used to treat epilepsy. This compound is quantified from human sample material to monitor its concentration post patient administration. The drug's therapeutic range is narrow and patient dosing has to be monitored to prevent toxic effects This clinical practice and the individualization of drug dosage my maintaining plasma/blood drug concentrations within a defined therapeutic range is called therapeutic drug monitoring (TDM).

Salicylic acid is a small molecule with a molecular weight of only 138 g/mol, which is the major, active metabolite of acetylsalicylic acid. Acetylsalicylic acid with its anti-inflammatory and antipyretic effects is used to treat various types of pain as well as fever. Furthermore it is used as inhibitor of blood coagulation. Especially for this indication, monitoring of blood/plasma levels post patient administration (TDM) is regarded as a valuable tool to monitor therapy efficacy.

Preferably, the small molecules, e.g. valproic acid or salicylic acid, are quantified by means of LC-MS/MS, making optimal use of the specificity of the tandem MS module that generates unique fragments from the native (i.e. intact) molecule/compound. However, due to its small size and simple chemical structure, generation of stable fragment ions of valproic acid and salicylic acid is poor, making their specific determination by means of tandem mass spectrometry a challenge.

To circumvent this problem, currently a pseudo Multiple Reaction Monitoring (MRM) is used. In this case “pseudo MRM” refers to the quantitation of these analytes via detection of the intact molecule (i.e. the molecule is not fragmented and the Q1 and Q3 quadrupoles of a triple quadrupole MS select for the same m/z). Thus, in case of valproic acid, Q1 and Q3 select for the intact species having a m/z of 143 in negative mode.

Although used and admitted, the negative aspect of this approach is that this leads to loss of
i) sensitivity due to a high background signal,
ii) specificity since no fragmentation is performed, and/or
iii) high risk of interferences, since quantitation is not performed via the fragmented product ion that is characteristic of a certain precursor ion, as is usually the case for TDM by means of MS/MS.

The latter may lead to over-estimation of the compound concentration in case there are interfering compounds with the same m/z present in either the sample material or one of the used materials needed in the sample preparation or LC-MS workflow, assuming the retention time is not significantly different from valproic acid.

Therefore, incorrect results and thus patient dosing may be the consequence of such an approach.

Therefore, there is an urgent need in the art to overcome the above mentioned problems.

It is an object of the present invention to provide a method for determining the presence or level of an analyte of interest and the use thereof. Further, it is an object of the present invention to provide an analytical system, a sampling tube and the use of the sampling tube and a nucleophilic derivatization reagent for determining the presence or level of an analyte of interest.

This object is or these objects are solved by the subject matter of the independent claims. Further embodiments are subjected to the dependent claims.

### SUMMARY OF THE INVENTION

In the following, the present invention relates to the following aspects:

In a first aspect, the present invention relates to a method for determining the presence or level of an analyte of interest having a molar mass of smaller than 200 Da in a sample comprising the steps of
a) Providing the sample comprising the analyte of interest, wherein the analyte of interest comprises a carboxylic acid group,
b) Optionally activating the analyte of interest by the addition of at least one activation reagent,
c) Derivatizing the analyte of interest provided by step a) orb) with a nucleophilic derivatization reagent for forming a derivatized analyte of interest, and
d) Determining the presence or level of the derivatized analyte of interest in the sample using immunological assay or mass spectrometry (MS).

In a second aspect, the present invention relates to an analytical system adapted to perform the method of the first aspect of the invention.

In a third aspect, the present invention relates to a sampling tube for collecting a patient sample comprising a nucleophilic derivatization reagent for forming a derivatized analyte of interest in a sample, wherein the one or more analytes of interests is a carboxylic acid having a molar mass of smaller than 200 Da, preferably wherein the one or more analytes of interests is valproic acid or salicylic acid.

In a fourth aspect, the present invention relates to the use of the sampling tube of the third aspect of the invention in a method according to the first aspect of the invention.

In a fifth aspect, the present invention relates to the use of a nucleophilic derivatization reagent in a method according to the first aspect of the invention.

In a sixth aspect, the present invention relates to the use of the method of the first aspect of the invention for determining the presence or level of one or more analytes of interest in a sample.

### LIST OF FIGURES

FIGS. 1 to 6 show chromatograms.

FIG. 1 shows valproic-butylamide from serum.

FIG. 2 shows valproic-butylamide from serum 1:10 diluted.

FIG. 3 shows valproic-propylamide from serum 1:10 diluted.

FIG. 4 shows valproic-propylamide from serum.

FIG. 5 shows Cal 3 (12 μg/mL) worked up from serum using enrichment workflow, detecting pseudo MRM 143.1/143.1 according to the prior art. S/N: 710.5

FIG. 6 shows Cal 3 (12 μg/mL) worked up from serum with derivatization followed by enrichment workflow, detecting MRM 200.1/116 according to the present invention. S/N: 2837.5

### DETAILED DESCRIPTION OF THE INVENTION

Before the present invention is described in detail below, it is to be understood that this invention is not limited to the particular embodiments and examples described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The various described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

The word “comprise”, and variations such as “comprises” and “comprising”, will be understood to imply the inclusion of a stated integer or step or group of integers or steps but not the exclusion of any other integer or step or group of integers or steps.

As used in this specification and the appended claims, the singular forms “a”, “an”, and “the” include plural referents, unless the content clearly dictates otherwise.

Percentages, concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “4% to 20%” should be interpreted to include not only the explicitly recited values of 4% to 20%, but to also include individual values and sub-ranges within the indicated range. Thus, included in this numerical range are individual values such as 4, 5, 6, 7, 8, 9, 10, . . . 18, 19, 20% and sub-ranges such as from 4-10%, 5-15%, 10-20%, etc. This same principle applies to ranges reciting minimal or maximal values. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

The term “measurement”, “measuring” or “determining” preferably comprises a qualitative, a semi-quanitative or a quantitative measurement. The term “determining” the presence or level of an analyte of interest, as used herein can be refer to the qualification or quantification of the analyte of interest, e.g. to determining or measuring the level of the analyte of interest in the sample, employing appropriate methods of detection described elsewhere herein.

In this context “level” or “level value” encompasses the absolute amount, the relative amount or concentration as well as any value or parameter which correlates thereto or can be derived therefrom.

The term “sample” or “patient sample” as used herein refers to a biological sample obtained for the purpose of evaluation in vitro. In the methods of the present invention, the sample or patient sample preferably may comprise any body fluid. The sample can include blood, serum, plasma, urine, saliva, and synovial fluid. Preferred samples are whole blood, serum or plasma. As the skilled artisan will appreciate, any such assessment is made in vitro. The patient sample is discarded afterwards. The patient sample is solely used for the in vitro method of the invention and the material of the patient sample is not transferred back into the patient's body.

In the context of the present disclosure, the term “analyte”, “analyte molecule” , or “analyte(s) of interest” are used interchangeably referring the chemical species to be analysed via mass spectrometry. Chemical species suitable to be analysed via mass spectrometry, i.e. analytes, can be any kind of molecule present in a living organism, include but are not limited to nucleic acid (e.g. DNA, mRNA, miRNA, rRNA etc.), amino acids, peptides, proteins (e.g. cell surface receptor, cytosolic protein etc.), metabolite or hormones (e.g. testosterone, estrogen, estradiol, etc.), fatty acids, lipids, carbohydrates, steroids, ketosteroids, secosteroids (e.g. Vitamin D), molecules characteristic of a certain modification of another molecule (e.g. sugar moieties or phosphoryl residues on proteins, methyl-residues on genomic DNA) or a substance that has been internalized by the organism (e.g. therapeutic drugs, drugs of abuse, toxins, etc.) or a metabolite of such a substance. The analyte has a molar mass of smaller than 200 Da (Dalton) or 200 g/mol, preferably smaller than 190 g/mol or 180 g/mol or 170 g/mol or 160 g/mol or 150 g/mol. Additionally, the analyte has a molar mass of more than 100 g/mol or 110 g/mol or 120 g/mol or 130 g/mol. Preferably, the analyte has a molar mass between 130 g/mol and 150 g/mol, preferably between 135 g/mol and 145 g/mol.

The term “molar mass” of the analyte of interest refers to the mass of a given chemical element or chemical compound (g) divided by the amount of substance (mol). The molar mass of a compound can be calculated by adding the standard atomic masses (in g/mol) of the constituent atoms. The unit of the molar mass can be kg/mole, g/mol or Da. Molar mass in g/mol is approximately numerically equal to the mass of one molecule in daltons (1 Da=1 u=1.66053906660(50)×10⁻²⁷ kg).

In the context of the present invention, the term “activation reagent” refers to a compound or mixture of compounds by which the carboxylic acid is activated, thereby rendering the carbonyl group of the analyte of interest susceptible for nucleophilic attack.

In the context of the present invention, the term “nucleophilic derivatization reagent” or “derivatization reagent” refers to a chemical substance having a specific chemical structure. Said derivatization reagent may comprise one or more reactive groups, which is or are capable of forming a bond, preferably a covalent bond, with the analyte of interest. A derivatized analyte of interest results. Each reactive group may fulfil a different functionality, or two or more reactive groups may fulfil the same function. Reactive groups include but are not limited to reactive units, charged units, and neutral loss units. In the context of the present invention, the term “nucleophilic” refers to a chemical species that donates an electron pair to form a chemical bond. Nucleophiles that exists in a water medium include but are not limited to —NH₂, —OH, —SH, —Se, (R′,R″,R′″)P, N₃—, RCOOH, F—, Cl—, Br—, I—. The term “nucleophilic derivatization reagent” can refer to reagents comprising such nucleophile. A nucleophilic derivatization reagent comprises a moiety, carrying an orbital that serves as the highest occupied molecular orbital (HOMO) that is able to attack the lowest unoccupied molecular orbital (LUMO) of the substance of interest, such as an analyte of interest, thereby forming a new molecule comprised of the formerly nucleophilic unit and the analyte moiety.

The term “derivatized analyte of interest” may refer to any molecule that is formed from the original analyte of interest and that is enlarged by a chemical reaction.

The term “analyte of interest comprises a carboxylic acid group” means that the analyte of interest has a carboxylic acid group as a functional group. The functional group can react with the nucleophilic derivatization reagent, preferably with a reactive group of the nucleophilic derivatization reagent. Carboxyl group and carboxylic acid group can be used in the content of the present invention interchangeable.

The term “automated” refers to methods or processes which are operated largely by automatic equipment, i.e. which are operated by machines or computers, in order to reduce the amount of work done by humans and the time taken to do the work. Thus, in an automated method, tasks that were previously performed by humans, are now performed by machines or computers. Typically, the users only need to configure the tool and define the process. The skilled person is well aware that at some minor points manual intervention may still be required, however the large extend of the method is performed automatically.

The term “analyte of interest does not show a characteristic fragmentation pattern in the mass spectrum compared to the derivatized analyte of interest” means, that the analyte of interest, the intact molecule, does not undergo any fragmentation due to stable molecular bonds. By enlarging the molecular mass through derivatization, thus forming the derivatized analyte of interest, the molecule get's more susceptible to fragmentation, which may lead to characteristic fragmentation patterns.

The term “Mass Spectrometry” (“Mass Spec” or “MS”) or “mass spectrometric determination” or “mass spectrometric analysis” relates to an analytical technology used to identify compounds by their mass. MS is a method of filtering, detecting, and measuring ions based on their mass-to-charge ratio, or “m/z”. MS technology generally includes (1) ionizing the compounds to form charged compounds; and (2) detecting the molecular weight of the charged compounds and calculating a mass-to-charge ratio. The compounds may be ionized and detected by any suitable means. A “mass spectrometer” generally includes an ion source and an ion detector. In general, one or more molecules of interest are ionized, and the ions are subsequently introduced into a mass spectrometric instrument where, due to a combination of magnetic and electric fields, the ions follow a path in space that is dependent upon mass (“m”) and charge (“z”). The term “ionization” or “ionizing” refers to the process of generating an analyte ion having a net charge equal to one or more units. Negative ions are those having a net negative charge of one or more units, while positive ions are those having a net positive charge of one or more units. The MS method may be performed either in “negative ion mode”, wherein negative ions are generated and detected, or in “positive ion mode” wherein positive ions are generated and detected. “After fragmentation by mass spectrometric determination” can mean that e.g. the compound, composition or complex passed through a mass spectrometer and were fragmented.

“Tandem mass spectrometry” or “MS/MS” involves multiple steps of mass spectrometry selection, wherein fragmentation of the analyte occurs in between the stages. In a tandem mass spectrometer, ions are formed in the ion source and separated by mass-to-charge ratio in the first stage of mass spectrometry (MSI). Ions of a particular mass-to-charge ratio (precursor ions or parent ion) are selected and fragment ions (or daughter ions) are created by collision-induced dissociation, ion-molecule reaction, or photodissociation. The resulting ions are then separated and detected in a second stage of mass spectrometry (MS2).

Since a mass spectrometer separates and detects ions of slightly different masses, it easily distinguishes different isotopes of a given element. Mass spectrometry is thus, an important method for the accurate mass determination and characterization of analytes, including but not limited to low-molecular weight analytes, peptides, polypeptides or proteins. Its applications include the identification of proteins and their post-translational modifications, the elucidation of protein complexes, their subunits and functional interactions, as well as the global measurement of proteins in proteomics. De novo sequencing of peptides or proteins by mass spectrometry can typically be performed without prior knowledge of the amino acid sequence.

Most sample workflows in MS further include sample preparation and/or enrichment steps, wherein e.g. the analyte(s) of interest are separated from the matrix using e.g. gas or liquid chromatography. Typically, for the mass spectrometric measurement, the following three steps are performed:
1. a sample comprising an analyte of interest is ionized, usually by complex formation with cations, often by protonation to cations. Ionization source include but are not limited to electrospray ionization (ESI) and atmospheric pressure chemical ionization (APCI).
2. the ions are sorted and separated according to their mass and charge. High-field asymmetric-waveform ion-mobility spectrometry (FAIMS) may be used as ion filter.
3. the separated ions are then detected, e.g. in multiple reaction mode (MRM), and the results are displayed on a chart.

The term “electrospray ionization” or “ESI,” refers to methods in which a solution is passed along a short length of capillary tube, to the end of which is applied a high positive or negative electric potential. Solution reaching the end of the tube is vaporized (nebulized) into a jet or spray of very small droplets of solution in solvent vapor. This mist of droplets flows through an evaporation chamber, which is heated slightly to prevent condensation and to evaporate solvent. As the droplets get smaller the electrical surface charge density increases until such time that the natural repulsion between like charges causes ions as well as neutral molecules to be released.

The term “atmospheric pressure chemical ionization” or “APCI,” refers to mass spectrometry methods that are similar to ESI; however, APCI produces ions by ion-molecule reactions that occur within a plasma at atmospheric pressure. The plasma is maintained by an electric discharge between the spray capillary and a counter electrode. Then ions are typically extracted into the mass analyzer by use of a set of differentially pumped skimmer stages. A counterflow of dry and preheated Ni gas may be used to improve removal of solvent. The gas-phase ionization in APCI can be more effective than ESE for analyzing less-polar entity.

“High-field asymmetric-waveform ion-mobility spectrometry (FAIMS)” is an atmospheric pressure ion mobility technique that separates gas-phase ions by their behavior in strong and weak electric fields.

“Multiple reaction mode” or “MRM” is a detection mode for a MS instrument in which a precursor ion and one or more fragment ions are selectively detected.

Mass spectrometric determination may be combined with additional analytical methods including chromatographic methods such as gas chromatography (GC), liquid chromatography (LC), particularly HPLC, and/or ion mobility-based separation techniques.

In the context of the present disclosure, the sample may be derived from an “individual” or “subject”. Typically, the subject is a mammal. Mammals include, but are not limited to, domesticated animals (e.g., cows, sheep, cats, dogs, and horses), primates (e.g., humans and non-human primates such as monkeys), rabbits, and rodents (e.g., mice and rats).

Before being analysed via Mass Spectrometry, a sample may be treated or pre-treated in a sample- and/or analyte specific manner. In the context of the present disclosure, the term “treatment” or “pre-treatment” refers to any measures required to allow for the subsequent analysis of a desired analyte via mass spectrometry. (Pre-)treatment measures typically include but are not limited to the elution of solid samples (e.g. elution of dried blood spots), addition of hemolizing reagent (HR) to whole blood samples, and the addition of enzymatic reagents to urine samples. Also the addition of internal standards (ISTD) is considered as pre-treatment of the sample.

The term “hemolysis reagent” (HR) refers to reagents which lyse cells present in a sample, in the context of this invention hemolysis reagents in particular refer to reagents which lyse the cell present in a blood sample including but not limited to the erythrocytes present in whole blood samples. A well known hemolysis reagent is water (H₂O). Further examples of hemolysis reagents include but are not limited to deionized water, liquids with high osmolarity (e.g. 8M urea), ionic liquids, and different detergents.

Typically, an “internal standard” (ISTD) is a known amount of a substance which exhibits similar properties as the analyte of interest when subjected to the mass spectrometric detection workflow (i.e. including any (pre-)treatment, enrichment and actual detection step). Although the ISTD exhibits similar properties as the analyte of interest, it is still clearly distinguishable from the analyte of interest. Exemplified, during chromatographic separation, such as gas or liquid chromatography, the ISTD has about the same retention time as the analyte of interest from the sample. Thus, both the analyte and the ISTD enter the mass spectrometer at the same time. The ISTD however, exhibits a different molecular mass than the analyte of interest from the sample. This allows a mass spectrometric distinction between ions from the ISTD and ions from the analyte by means of their different mass/charge (m/z) ratios. Both are subject to fragmentation and provide daughter ions. These daughter ions can be distinguished by means of their m/z ratios from each other and from the respective parent ions. Consequently, a separate determination and quantification of the signals from the ISTD and the analyte can be performed. Since the ISTD has been added in known amounts, the signal intensity of the analyte from the sample can be attributed to a specific quantitative amount of the analyte. Thus, the addition of an ISTD allows for a relative comparison of the amount of analyte detected, and enables unambiguous identification and quantification of the analyte(s) of interest present in the sample when the analyte(s) reach the mass spectrometer. Typically, but not necessarily, the ISTD is an isotopically labeled variant (comprising e.g. ²H, ¹³C, or ¹⁵N etc. label) of the analyte of interest.

In addition to the pre-treatment, the sample may also be subjected to one or more enrichment steps. In the context of the present disclosure, the term “first enrichment process” or “first enrichment workflow” refers to an enrichment process which occurs subsequent to the (pre-)treatment of the sample and provides a sample comprising an enriched analyte relative to the initial sample. The first enrichment workflow may comprise chemical precipitation (e.g. using acetonitrile) or the use of a solid phase. Suitable solid phases include but are not limited to Solid Phase Extraction (SPE) cartridges, and beads. Beads may be non-magnetic, magnetic, paramagnetic or supermagnetic. Beads may be coated differently to be specific for the analyte of interest. The coating may differ depending on the use intended, i.e. on the intended capture molecule. It is well-known to the skilled person which coating is suitable for which analyte. The beads may be made of various different materials. The beads may have various sizes and comprise a surface with or without pores.

In the context of the present disclosure the term “second enrichment process” or “second enrichment workflow” refers to an enrichment process which occurs subsequent to the (pre-)treatment and the first enrichment process of the sample and provides a sample comprising an enriched analyte relative to the initial sample and the sample after the first enrichment process.

The term “chromatography” refers to a process in which a chemical mixture carried by a liquid or gas is separated into components as a result of differential distribution of the chemical entities as they flow around or over a stationary liquid or solid phase.

The term “liquid chromatography” or “LC” refers to a process of selective retardation of one or more components of a fluid solution as the fluid uniformly percolates through a column of a finely divided substance, or through capillary passageways. The retardation results from the distribution of the components of the mixture between one or more stationary phases and the bulk fluid, (i.e., mobile phase), as this fluid moves relative to the stationary phase(s). Methods in which the stationary phase is more polar than the mobile phase (e.g., toluene as the mobile phase, silica as the stationary phase) are termed normal phase liquid chromatography (NPLC) and methods in which the stationary phase is less polar than the mobile phase (e.g., water-methanol mixture as the mobile phase and C18 (octadecylsilyl) as the stationary phase) is termed reversed phase liquid chromatography (RPLC).

“High performance liquid chromatography” or “HPLC” refers to a method of liquid chromatography in which the degree of separation is increased by forcing the mobile phase under pressure through a stationary phase, typically a densely packed column. Typically, the column is packed with a stationary phase composed of irregularly or spherically shaped particles, a porous monolithic layer, or a porous membrane. HPLC is historically divided into two different sub-classes based on the polarity of the mobile and stationary phases. Methods in which the stationary phase is more polar than the mobile phase (e.g., toluene as the mobile phase, silica as the stationary phase) are termed normal phase liquid chromatography (NPLC) and the opposite (e.g., water-methanol mixture as the mobile phase and C18 (octadecylsilyl) as the stationary phase) is termed reversed phase liquid chromatography (RPLC). Micro LC refers to a HPLC method using a column having a narrow inner column diameter, typically below 1 mm, e.g. about 0.5 mm. “Ultra high performance liquid chromatography” or “UHPLC” refers to a HPLC method using a pressure of 120 MPa (17,405 lbf/in2), or about 1200 atmospheres. Rapid LC refers to an LC method using a column having an inner diameter as mentioned above, with a short length <2 cm, e.g. 1 cm, applying a flow rate as mentioned above and with a pressure as mentioned above (Micro LC, UHPLC). The short Rapid LC protocol includes a trapping/wash/elution step using a single analytical column and realizes LC in a very short time <1 min.

Further well-known LC modi include hydrophilic interaction chromatography (HILIC), size-exclusion LC, ion exchange LC, and affinity LC.

LC separation may be single-channel LC or multi-channel LC comprising a plurality of LC channels arranged in parallel. In LC analytes may be separated according to their polarity or log P value, size or affinity, as generally known to the skilled person.

The term “sampling tube” or “sample collection tube” refers to any device with a reservoir appropriate for receiving sample, e.g. a blood sample to be collected.

### Embodiments

In a first aspect, the present invention relates to a method for determining the presence or level of an analyte of interest having a molar mass of smaller than 200 Da in a sample comprising the steps of
a) Providing the sample comprising the analyte of interest, wherein the analyte of interest comprises a carboxylic acid group,
b) Optionally activating the analyte of interest by the addition of at least one activation reagent,
c) Derivatizing the analyte of interest provided by step a) orb) with a nucleophilic derivatization reagent for forming a derivatized analyte of interest, and
d) Determining the presence or level of the derivatized analyte of interest in the sample using immunological assay or mass spectrometry (MS).

In embodiments of the first aspect of the present invention, the method is automated. In particular embodiments, the method is performed by an automated system. In particular embodiments, the method comprises no manual intervention.

According to step (a), the sample comprising the analyte of interest is provided. The analyte of interest comprises a carboxylic acid group. Preferably, the analyte of interest is a carboxylic acid.

In embodiments of the first aspect of the present invention, the analyte of interest comprises a carboxylic acid group and does not comprise a reactive group, which is capable of reacting with carboxylic acid group of the analyte of interest. The reactive group can be an amine, e.g. primary amine.

In embodiments of the first aspect of the present invention, the analyte of interest comprises a carboxylic acid group and does not comprise a reactive group, which is capable of an intramolecular reaction of the analyte of interest.

In embodiments of the first aspect of the present invention, analyte of interest is free of a nucleophilic functional groups, which could react with the carboxylic acid group of the analyte of interest.

In embodiments of the first aspect of the present invention, the analyte of interest is valproic acid or salicylic acid.

In embodiments of the first aspect of the present invention, the analyte of interest is free of pregabalin or the analyte of interest is not pregabalin.

In embodiments of the first aspect of the present invention, analyte of interest has a molar mass of 150 Da or less.

In embodiments of the first aspect of the present invention, the analyte of interest has a molar mass of smaller than 200 Da (Dalton) or 200 g/mol, preferably smaller than 190 g/mol or 180 g/mol or 170 g/mol or 160 g/mol or 150 g/mol. Additionally, the analyte has a molar mass of more than 100 g/mol or 110 g/mol or 120 g/mol or 130 g/mol. Preferably, the analyte has a molar mass between 130 g/mol and 150 g/mol, preferably between 135 g/mol and 145 g/mol.

Optionally a step (b) can be performed, activating the analyte of interest by the addition of at least one activation reagent. The activation reagent can be at least one activation reagent or a mixture of activation reagents.

In embodiments of the first aspect of the present invention, the least one activation reagent is a first activation reagent or a second activation reagent or a combination thereof,

wherein the first activation reagent is selected from the group consisting of N-Hydroxysuccinimid (OHSu), N-Hydroxysulfosuccinimide (sulfo-OSu), Hydroxybenzotriazole (HOBt) and salts of these compounds,

wherein the second activation reagent is selected from the group consisting of Dicyclohexylcarbodiimid (DIC), 1-Ethyl-3-(3-dimethylaminopropyl)carbodiimide (EDC), N-(3-Dimethylaminopropyl)-N′-ethylcarbodiimide, N,N′-Dicyclohexylcarbodiimide, N-Cyclohexyl-N′-(2-morpholinoethyl)carbodiimide methyl-p-toluenesulfonate, 1,3-Bis(trimethylsilyl)carbodiimid and N,N′-Methanetetraylbis[4-methyl]benzenamine.

In principle, every suitable activation reagent can be used, which is used in peptide chemistry for the formation of amide bonds.

In embodiments of the first aspect of the present invention, an ester of the analyte of interest is formed in step (b).

According to step (c), the analyte of interest provided by step a) or b) is derivatized with a nucleophilic derivatization reagent for forming a derivatized analyte of interest.

In embodiments of the first aspect of the present invention, in step c) an amide of the derivatized analyte of interest is formed.

In embodiments of the first aspect of the present invention, the molar mass of the derivatized analyte of interest provided after step c) is greater than the molar mass of the analyte of interest provided before step c).

In embodiments of the first aspect of the present invention, the analyte of interest does not show a characteristic fragmentation pattern in the mass spectrum compared to the derivatized analyte of interest. Since the analyte of interest has a small molecular mass combined with stable molecular bonds, low to no fragmentation can be achieved within the collision cell. By enlarging the molecular mass through derivatization, the derivatized molecule get's more susceptible to fragmentation, thus leading to a characteristic fragmentation pattern.

In embodiments of the first aspect of the present invention, the nucleophilic derivatization reagent comprises an amine group, in particular a primary amine group or secondary amine group or tertiary amine group, more in particular a secondary amine group, more in particular a primary amine group group.

In embodiments of the first aspect of the present invention, the nucleophilic derivatization reagent comprises 1 C-atom, in particular 3 to 20 C-atoms, in particular 3 to 10 C-atoms, in particular 3 to 5 C-atoms, in particular 4 C-atoms.

In embodiments of the first aspect of the present invention, the nucleophilic derivatization reagent is selected from the group consisting of methylamine, ethylamine, butylamine, n-propylamine, isopropylamine pentylamine, hexylamine

In embodiments, the nucleophilic derivatization reagent comprises an amine group, in particular a primary or secondary amine, in particular a primary amine group. In embodiments, the nucleophilic derivatization reagent comprises more than 3 C-atoms, in particular 3 to 20 C-atoms, in particular 3 to 10 C-atoms, in particular 3 to 5 C-atoms, in particular 4 C-atoms. In embodiments, the nucleophilic derivatization reagent is linear or branched, in particular with a linear amine, in particular with a linear primary amine, in particular with a linear primary amine comprising 3 to 5 C-atoms.

In embodiments, the nucleophilic derivatization reagent is selected from the group consisting of propylamine, butylamine, or pentylamine, in particular primary linear butylamine or primary linear pentylamine.

In embodiments, the nucleophilic derivatization reagent derivatizes the antibiotic analyte in at least one of its chemical moieties. The person skilled in the art of chemistry is well-aware of chemical moieties which are suitable to be derivatized, in particular with a nucleophilic derivatization reagent.

In embodiments of the first aspect of the present invention, the nucleophilic derivatization reagent is selected from the group consisting of propylamine, butylamine, in particular primary linear butylamine.

In embodiments of the first aspect of the present invention, the nucleophilic derivatization reagent is a linear or branched nucleophilic derivatization reagent, in particular a linear amine, in particular a linear primary amine, in particular a linear primary amine comprising 3 to 5 C-atoms.

In embodiments of the first aspect of the present invention, the said method comprises an additional step:
e) Enriching the sample, in particular using magnetic beads.

In embodiments of the first aspect of the present invention, the enrichment step e) comprises using magnetic beads, in particular type A or B magnetic beads.

In embodiments of the first aspect of the present invention, the enrichment step e) comprises at least one enrichment workflow.

In embodiments of the first aspect of the present invention, enrichment step a) comprises two enrichments steps, in particular a first enrichment step comprising using magnetic beads, and a second enrichment step using evaporation.

The sample may be further subjected to at least one enrichment workflow. The enrichment workflow may include one or more enrichment methods. Enrichment methods are well-known in the art and include but are not limited to chemical enrichment methods including but not limited to chemical precipitation, and enrichment methods using solid phases including but not limited to solid phase extraction methods, bead workflow's, and chromatographic methods (e.g. gas or liquid chromatography). It is also well-known to the skilled person which enrichment method is suitable for which analyte of interest.

In embodiments of the first aspect of the present invention, a first enrichment workflow comprises the addition of a solid phase, in particular of solid beads, carrying analyte-selective groups, to the sample. In embodiments, a first enrichment workflow comprises the addition of magnetic or paramagnetic beads carrying analyte-selective groups to the pre-treated sample.

In embodiments of the first aspect of the present invention, the magnetic beads comprise a magnetic core coated with a styrene based polymer that is hypercrosslinked via Friedel-Crafts alkylation and further modified with addition of —OH groups.

In embodiments of the first aspect of the present invention, the magnetic beads, preferably of bead type B, comprise a magnetic core coated with a styrene based polymer that is hypercrosslinked via diamines (e.g. TMEDA) and further modified whereby the diamine also serves as a sidechain (i.e. in these types of beads, TMEDA offers both quaternary and tertiary amine functionalities). For a full description of such beads see e.g. WO 2019/141779 A 1.

In embodiments of the first aspect of the present invention, the addition of the magnetic beads comprises agitation or mixing. A pre-defined incubation period for capturing the analyte(s) of interest on the bead follows. In embodiments, the workflow comprises a washing step (W1) after incubation with the magnetic beads. Depending on the analyte(s) one or more additional washing steps (W2) are performed. One washing step (W1, W2) comprises a series of steps including magnetic bead separation by a magnetic bead handling unit comprising magnets or electromagnets, aspiration of liquid, addition of a washing buffer, resuspension of the magnetic beads, another magnetic bead separation step and another aspiration of the liquid. Moreover, washing steps may differ in terms of type of solvent (water/organic/salt/pH), aside from volume and number or combination of washing cycles. It is well-known to the skilled person how to choose the respective parameters. The last washing step (W1, W2) is followed by the addition of an elution reagent followed by resuspension of the magnetic beads and a pre-defined incubation period for releasing the analyte(s) of interest from the magnetic beads. The bound-free magnetic beads are then separated and the supernatant containing derivatized analyte(s) of interest is captured.

In embodiments of the first aspect of the present invention, a first enrichment workflow comprises the addition of magnetic beads carrying matrix-selective groups to the pre-treated sample. In embodiments, the addition of the magnetic beads comprises agitation or mixing. A pre-defined incubation period for capturing the matrix on the bead follows. Here, the analyte of interest does not bind to the magnetic beads but remains in the supernatant. Thereafter, the magnetic beads are separated and the supernatant containing the enriched analyte(s) of interest is collected.

In embodiments of the first aspect of the present invention, the supernatant is subjected to a second enrichment workflow, in particular to a chromatographic enrichment workflow. In embodiments of the present invention, the chromatographic separation is gas or liquid chromatography. Both methods are well known to the skilled person. In embodiments, the liquid chromatography is selected from the group consisting of HPLC, rapid LC, micro-LC, flow injection, and trap and elute. Here, the supernatant is transferred to the LC station or is transferred to the LC station after a dilution step by addition of a dilution liquid. Different elution procedures/reagents may also be used, by changing e.g. the type of solvents (water/organic/salt/pH) and volume. The various parameters are well-known to the skilled person and easily chosen.

In embodiments of the first aspect of the present invention, the first enrichment process includes the use of analyte selective magnetic beads. In embodiments, the second enrichment process includes the use of chromatographic separation, in particular using liquid chromatography. In embodiments, the first enrichment process using analyte selective magnetic beads is performed prior to the second enrichment process using liquid chromatography.

In embodiments of the first aspect of the present invention, the enrichment step (e) is performed after step (c) and before step (d).

In embodiments of the first aspect of the present invention, before step d) a chromatographic step is performed, in particular liquid chromatography (LC).

In embodiments of the first aspect of the present invention, the chromatographic step and step d) are combined or are performed one after the other by using LC-MS, wherein the LC is HPLC, in particular RP-HPLC, or wherein LC is rapid LC.

In embodiments of the first aspect of the present invention, the chromatographic step and step d) are combined or are performed one after the other by using LC-MS, wherein the ion formation is based on electrospray ionization (ESI), in particular positive polarity mode ESI.

In embodiments of the first aspect of the present invention, the chromatographic step and step d) are combined or are performed one after the other by using LC-MS, wherein the mass spectrometry is performed by a mass device, wherein the mass device is a tandem mass spectrometer, in particular a triple quadrupole device, in particular an automated, random-access LC-MS device.

In embodiments of the first aspect of the present invention, step c) comprises a solvent, in particular a solvent selected from the group consisting of water, CH₃CN, THF, Dioxanes, DMF, DMSO, acetone, t-butyl alcohol, diglyme, DME, MeOH, EtOH, 1-PrOH, 2-PrOH, ethylene glycol, Hexamethylphosphoramide (HMPA), Hexamethylphosphorous triamide (HMPT), and glycerin, in particular a solvent selected from the group consisting of water, CH₃CN, THF, Dioxanes, DMF, DMSO, acetone, tBuOH, diglyme, and DME.

In embodiments of the first aspect of the present invention, step c) comprises a non-nucleophilic base that is stable and miscible with water, in particular selected from the group consisting of DBU, TEA, DIPEA, Na₃PO₄, Na₂CO₃, and Cs₂CO₃.

In embodiments of the first aspect of the present invention, in step b) the sample is treated with a activation reagent sample for more than 30 seconds, in particular more than 1 min, in particular more than 2 min.

In embodiments of the first aspect of the present invention, in step c) the sample is treated with the nucleophilic derivatization reagent immediately after the sample is obtained, in particular within less than 10 min after the sample was obtained, in particular within less than 5 min or 3 min. after the sample was obtained.

In embodiments of the first aspect of the present invention, in step c) the sample is treated with a nucleophilic derivatization reagent sample for more than 30 seconds, in particular more than 1 min, in particular more than 2 min.

According to step (d), the presence or level of the derivatized analyte of interest in the sample is determined using immunological assay or mass spectrometry (MS).

In embodiments of the first aspect of the present invention, step (d) comprises determining the presence or level of the one or more analyte using immunological methods, the following steps are comprised:
i) incubating the (optionally enriched) sample of the patient with one or more antibodies specifically binding to the one or more derivatized analyte, thereby generating a complex between the antibody and the one or more derivatized analyte, and
ii) quantifying the complex formed in step i), thereby quantifying the amount of the one or more derivatized analyte in the sample of the patient.

In particular embodiments, in step i) the sample is incubated with two antibodies, specifically binding to the one or more derivatized analyte. As obvious to the skilled artisan, the sample can be contacted with the first and the second antibody in any desired order, i.e. first antibody first and then the second antibody or second antibody first and then the first antibody, or simultaneously, for a time and under conditions sufficient to form a first antibody/derivatized analyte/second antibody complex. As the skilled artisan will readily appreciate it is nothing but routine experimentation to establish the time and conditions that are appropriate or that are sufficient for the formation of a complex either between the specific antibody and the derivatized analyte or the formation of the secondary, or sandwich complex comprising the first antibody, the derivatized analyte, the second antibody.

The detection of the antibody-analyte complex can be performed by any appropriate means. The person skilled in the art is absolutely familiar with such means/methods. In embodiments of the first aspect of the present invention, the antibody/the antibodies is/are directly or indirectly detectably labeled. In particular embodiments, the antibody is detectably labeled with a luminescent dye, in particular a chemiluminescent dye or an electrochemiluminescent dye.

In embodiments of the first aspect of the present invention, step (d) comprises determining the presence or the level of one or more derivatized antibiotic analyte using mass spectrometry, the following steps are comprised:
(i) subjecting an ion of the derivatized analyte to a first stage of mass spectrometric analysis, whereby the parent ion of the derivatized analyte is characterised according to its mass/charge (m/z) ratio,
(ii) causing fragmentation of the derivatized analyte parent ion, whereby a daughter ion is generated, wherein the daughter ion of the derivatized analyte differs in its m/z ratio from the derivatized analyte parent ion, and
(iii) subjecting the daughter ion of the derivatized analyte to a second stage of mass spectrometric analysis, whereby the daughter ion of the derivatized analyte is characterized according to its m/z ratio.

In embodiments, the parent and/or fragment ions measured are those as indicated in table X.

TABLE X
MRM transitions of valproic-amide
compounds and salicylic-amide compounds:

| | Parent ion | Fragment ion |
|---|---|---|
| | (Q1 Mass) | (Q3 Mass) |
| Analyte | (Da) | (Da) |
| Valproic-butylamide MRM 1 | 200.098 | 116 |
| Valproic-butylamide MRM 2 | 200.098 | 60.1 |
| Valproic-butylamide MRM 3 | 200.098 | 57 |
| Valproic-butylamide-D3 MRM 1 | 203.098 | 119 |
| Valproic-butylamide-D3 MRM 2 | 203.098 | 63.1 |
| Valproic-butylamide-D3 MRM 3 | 203.098 | 60 |
| Salicylic-butylamide MRM 1 | 191.87 | 92.2 |
| Salicylic-butylamide MRM 2 | 191.87 | 74.9 |
| Salicylic-butylamide MRM 3 | 191.87 | 65 |
| Salicylic-butylamide-D4 MRM 1 | 195.881 | 97.1 |
| Salicylic-butylamide-D4 MRM 2 | 195.881 | 79.2 |
| Salicylic-butylamide-D4 MRM 3 | 195.881 | 69.2 |

In embodiments of the first aspect of the present invention, in step d) the present or level of the derivatized analyte of interest is determined by LC-MS, wherein the parent ion of derivatized valproic butylamide (N-butyl-2-propylpentanamide) is measured at a m/z value 200.2±1 in positive mode, and/or the parent ion of derivatized salicylic butylamide (2-hydroxy-N-butylbenzamide) is measured at a m/z value 191.87±1 in negative mode.

In a second aspect, the present invention relates to an analytical system adapted to perform the method of the first aspect of the invention.

All embodiments mentioned for the first aspect of the invention apply for the second aspect of the invention and vice versa.

In embodiments of the second aspect of the present invention, the analytical system is a mass spectrometry system, in particular an LC-MS system.

In embodiments of the second aspect of the present invention, the analytical system is automated. In particular embodiments, the analytical system does not require manual intervention, i.e. the operation of the system is purely automated. In particular embodiments, the LC-MS system is an automated, random-access LC-MS system.

In embodiments of the second aspect of the present invention, the MS device is a tandem mass spectrometer, in particular a triple quadrupole device. In embodiments, the LC is HPLC, in particular is RP-HPLC, or rapid LC.

In embodiments of the second aspect of the present invention, the ion formation is based on electrospray ionization (ESI) or atmospheric pressure chemical ionization (APCI), in particular positive polarity mode ESI.

In embodiments of the second aspect of the present invention, the analytical system is a clinical diagnostics system.

A “clinical diagnostics system” is a laboratory automated apparatus dedicated to the analysis of samples for in vitro diagnostics. The clinical diagnostics system may have different configurations according to the need and/or according to the desired laboratory workflow. Additional configurations may be obtained by coupling a plurality of apparatuses and/or modules together. A “module” is a work cell, typically smaller in size than the entire clinical diagnostics system, which has a dedicated function. This function can be analytical but can be also pre-analytical or post analytical or it can be an auxiliary function to any of the pre-analytical function, analytical function or post-analytical function. In particular, a module can be configured to cooperate with one or more other modules for carrying out dedicated tasks oaf sample processing workflow, e.g. by performing one or more pre-analytical and/or analytical and/or post-analytical steps. In particular, the clinical diagnostics system can comprise one or more analytical apparatuses, designed to execute respective workflows that are optimized for certain types of analysis, e.g. clinical chemistry, immunochemistry, coagulation, hematology, liquid chromatography separation, mass spectrometry, etc. Thus the clinical diagnostic system may comprise one analytical apparatus or a combination of any of such analytical apparatuses with respective workflows, where pre-analytical and/or post analytical modules may be coupled to individual analytical apparatuses or be shared by a plurality of analytical apparatuses. In alternative pre-analytical and/or post-analytical functions may be performed by units integrated in an analytical apparatus. The clinical diagnostics system can comprise functional units such as liquid handling units for pipetting and/or pumping and/or mixing of samples and/or reagents and/or system fluids, and also functional units for sorting, storing, transporting, identifying, separating, detecting. The clinical diagnostic system can comprise a sample preparation station for the automated preparation of samples comprising analytes of interest, optionally a liquid chromatography (LC) separation station comprising a plurality of LC channels and/or optionally a sample preparation/LC interface for inputting prepared samples into any one of the LC channels. The clinical diagnostic system can further comprise a controller programmed to assign samples to pre-defined sample preparation workflows each comprising a pre-defined sequence of sample preparation steps and requiring a pre-defined time for completion depending on the analytes of interest. The clinical diagnostic system can further comprise a mass spectrometer (MS) and an LC/MS interface for connecting the LC separation station to the mass spectrometer. The term “automatically” or “automated” as used herein is a broad term and is to be given its ordinary and customary meaning to a person of ordinary skill in the art and is not to be limited to a special or customized meaning. The term specifically may refer, without limitation, to a process which is performed completely by means of at least one computer and/or computer network and/or machine, in particular without manual action and/or interaction with a user.

In embodiments of at least one aspect or all aspects of the present invention, the clinical diagnostic system comprises a sample preparation station.

A “sample preparation station” can be a pre-analytical module coupled to one or more analytical apparatuses or a unit in an analytical apparatus designed to execute a series of sample processing steps aimed at removing or at least reducing interfering matrix components in a sample and/or enriching analytes of interest in a sample. Such processing steps may include any one or more of the following processing operations carried out on a sample or a plurality of samples, sequentially, in parallel or in a staggered manner: pipetting (aspirating and/or dispensing) fluids, pumping fluids, mixing with reagents, incubating at a certain temperature, heating or cooling, centrifuging, separating, filtering, sieving, drying, washing, resuspending, aliquoting, transferring, storing, etc.).

The clinical diagnostic system, e.g. the sample preparation station, may also comprise a buffer unit for receiving a plurality of samples before a new sample preparation start sequence is initiated, where the samples may be individually randomly accessible and the individual preparation of which may be initiated according to the sample preparation start sequence.

The clinical diagnostic system makes use of mass spectrometry more convenient and more reliable and therefore suitable for clinical diagnostics. In particular, high-throughput, e.g. up to 100 samples/hour or more with random access sample preparation and LC separation can be obtained while enabling online coupling to mass spectrometry. Moreover the process can be fully automated increasing the walk-away time and decreasing the level of skills required.

The inventors surprisingly found that the said method can be implemented in the fully automated device, e.g. a cobas i601 analyzer (serum work area solution). This can mean that there is a soft analyte release step followed by immunobead capturing and detection by means of LC-MS/MS.

In a third aspect, the present invention relates to a sampling tube for collecting a patient sample comprising a nucleophilic derivatization reagent for forming a derivatized analyte of interest in a sample, wherein the one or more analytes of interests is a carboxylic acid having a molar mass of smaller than 200 Da, preferably wherein the one or more analytes of interests is valproic acid or salicylic acid.

All embodiments mentioned for the first aspect of the invention and/or second aspect of the invention apply for the third aspect of the invention and vice versa.

Sampling tubes suitable to be used for collecting a patient sample are well-known in the art and are used on a routine basis by practioners. As the skilled artisan will appreciate the sampling tube preferably will in fact be a tube. In particular, the sampling tube has a size and dimension adapted to match the requirements of the sample receiving station of an automated analyzer, e.g. an Elecsys® analyzer of Roche Diagnostics. The sampling tube may have a conical or preferably a round bottom. In clinical routine standard tube sizes are used that are compatible with the analyzers systems on the market. Standard and preferred tubes e.g. have the following dimensions: 13×75 mm; 13×100 mm, or 16×100 mm.

In embodiments of the third aspect of the present invention, the sampling tube according to the present invention is only used once, i.e. it is a single use device. In particular embodiments, the sampling tube according to the present invention is not only appropriate for collection of a sample but it is also adapted to allow for the further processing of the sample. By collecting a sample into a sampling tube containing the nucleophilic derivatization reagent, the desired result, i.e. the derivatization of the antibiotic analyte, is achieved.

In embodiments of the third aspect of the present invention, the sampling tube comprises a device with a reservoir adapted for receiving a sample to be collected. The sample can be a fluid sample such as blood, serum, plasma, synovial fluid, spinal fluid, urine, saliva, and lymphatic fluid, or solid sample such as dried blood spots and tissue extracts. Preferably, the sample is a blood sample.

In embodiments of the third aspect of the present invention, the nucleophilic derivatization reagent comprising an amine group, in particular a primary or secondary amine group, in particular a primary amine group.

In embodiments of the third aspect of the present invention, the nucleophilic derivatization reagent comprises 1 C-atom, in particular 3 to 20 C-atoms, in particular 3 to 10 C-atoms, in particular 3-5 C-atoms, in particular 4 C-atoms.

In embodiments of the third aspect of the present invention, the nucleophilic derivatization reagent is linear or branched amine, in particular a linear amine, in particular a linear primary amine, in particular a linear primary amine comprising 3 to 5 C-atoms.

In embodiments of the third aspect of the present invention, the nucleophilic derivatization reagent is selected from the group consisting of propylamine, butylamine, or pentylamine, in particular primary linear butylamine.

In embodiments of the third aspect of the present invention, the nucleophilic derivatization reagent is comprised in liquid or lyophilized form.

In embodiments of the third aspect of the present invention, the nucleophilic derivatization reagent further comprises a non-nucleophilic base that is stable and miscibile with water, in particular selected from the group consisting of DBU, TEA, DIPEA, Na₃PO₄, Na₂CO₃, and Cs₂CO₃.

In embodiments of the third aspect of the present invention, the nucleophilic derivatization reagent is embodied in liquid form in a solvent, in particular a solvent selected from the group consisting of water, CH3CN, THF, Dioxanes, DMF, DMSO, acetone, tBuOH, diglyme, DME, MeOH, EtOH, 1-PrOH, 2-PrOH, ethylene glycol, Hexamethylphosphoramide (HMPA), Hexamethylphosphorous triamide (HMPT), and glycerin, in particular a solvent selected from the group consisting of water, CH3CN, THF, Dioxanes, DMF, DMSO, acetone, tBuOH, diglyme, and DME.

In a fourth aspect, the present invention relates to the use of the sampling tube of the third aspects of the invention in a method according to the first aspect of the invention. All embodiments mentioned for the first aspect of the invention and/or second aspect of the invention and/or third aspect of the invention apply for the fourth aspect of the invention and vice versa.

In a fifth aspect, the present invention relates to the use of a nucleophilic derivatization reagent in a method according to the first aspect of the invention.

All embodiments mentioned for the first aspect of the invention and/or second aspect of the invention and/or third aspect of the invention and/or fourth aspect of the invention apply for the fifth aspect of the invention and vice versa.

In a sixth aspect, the present invention relates to the use of the method of the first aspect of the invention for determining the presence or level of one or more analytes of interest in a sample.

All embodiments mentioned for the first aspect of the invention and/or second aspect of the invention and/or third aspect of the invention and/or fourth aspect of the invention and/or fifth aspect of the invention apply for the sixth aspect of the invention and vice versa.

In further embodiments, the present invention relates to the following aspects:
1. A method for determining the presence or level of an analyte of interest having a molar mass of smaller than 200 Da in a sample comprising the steps of
   a) Providing the sample comprising the analyte of interest, wherein the analyte of interest comprises a carboxylic acid group,
   b) Optionally activating the analyte of interest by the addition of at least one activation reagent,
   c) Derivatizing the analyte of interest provided by step a) or b) with a nucleophilic derivatization reagent for forming a derivatized analyte of interest, and
   d) Determining the presence or level of the derivatized analyte of interest in the sample using immunological assay or mass spectrometry (MS).
2. The method of aspect 1, wherein the method is automated.
3. The method of aspect 1 or 2, wherein the analyte of interest has a molar mass of 150 Da or less.
4. The method of any of the proceeding aspects, wherein the analyte of interest is valproic acid or salicylic acid.
5. The method of any of the proceeding aspects, wherein the analyte of interest is not pregabalin or is free of pregabalin.
6. The method of any of the proceeding aspects, wherein in step c) an amide of the derivatized analyte of interest is formed.
7. The method of any of the proceeding aspects, wherein the molar mass of the derivatized analyte of interest provided after step c) is greater than the molar mass of the analyte of interest provided before step c).
8. The method of any of the proceeding aspects, wherein the analyte of interest does not show a characteristic fragmentation pattern in the mass spectrum compared to the derivatized analyte of interest.
9. The method of any of the proceeding aspects, wherein the nucleophilic derivatization reagent comprises an amine group, in particular a primary amine group or secondary amine group or tertiary amine group, more in particular a secondary amine group, more in particular a primary amine group.
10. The method of any of the proceeding aspects, wherein nucleophilic derivatization reagent comprises 1 C-atom, in particular 3 to 20 C-atoms, in particular 3 to 10 C-atoms, in particular 3 to 5 C-atoms, in particular 4 C-atoms.
11. The method of any of the proceeding aspects, wherein the nucleophilic derivatization reagent is a linear or branched nucleophilic derivatization reagent, in particular a linear amine, in particular a linear primary amine, in particular a linear primary amine comprising 3 to 5 C-atoms.
12. The method of any of the proceeding aspects, wherein the nucleophilic derivatization reagent is selected from the group consisting of propylamine, butylamine, or pentylamine, in particular primary linear butylamine.
13. The method of any of the proceeding aspects, wherein the at least one activation reagent is a first activation reagent or a second activation reagent or a combination thereof,
   wherein the first activation reagent is selected from the group consisting of N-Hydroxysuccinimid (OHSu), N-Hydroxysulfosuccinimide (sulfo-OSu), Hydroxybenzotriazole (HOBt) and salts of these compounds,
   wherein the second activation reagent is selected from the group consisting of Dicyclohexylcarbodiimid (DIC), 1-Ethyl-3-(3-dimethylaminopropyl)carbodiimid (EDC), N-(3-Dimethylaminopropyl)-N′-ethylcarbodiimide, N,N′-Dicyclohexylcarbodiimide, N-Cyclohexyl-N′-(2-morpholinoethyl)carbodiimide methyl-p-toluenesulfonate, 1,3-Bis(trimethylsilyl)carbodiimide and N,N′-Methanetetraylbis[4-methyl]benzenamine.
14. The method of any of the proceeding aspects, wherein the analyte of interest is free of a nucleophilic functional groups, which could react with the carboxylic acid group of the analyte of interest.
15. The method of any of the proceeding aspects, wherein the said method comprises an additional step:
   e) Enriching the sample, in particular using magnetic beads.
16. The method of any of the proceeding aspects, wherein the enrichment step e) comprises using magnetic beads, in particular type A or B magnetic beads.
17. The method of any of the proceeding aspects, wherein enrichment step e) comprises at least one enrichment workflow.
18. The method of any of the proceeding aspects, wherein enrichment step a) comprises two enrichments steps, in particular a first enrichment step comprising using magnetic beads, and a second enrichment step using evaporation.
19. The method of any of the proceeding aspects, wherein before step d) a chromatographic step is performed, in particular liquid chromatography (LC).
20. The method of any of the proceeding aspects, wherein the chromatographic step and step d) are combined or are performed one after the other by using LC-MS, wherein the EC is HPLC, in particular RP-HPLC, or wherein LC is rapid LC.
21. The method of any of the proceeding aspects, wherein the chromatographic step and step d) are combined or are performed one after the other by using LC-MS, wherein the ion formation is based on electrospray ionization (ESI), in particular positive polarity mode ESI.
22. The method of any of the proceeding aspects, wherein the chromatographic step and step d) are combined or are performed one after the other by using LC-MS, wherein the mass spectrometry is performed by a mass device, wherein the mass device is a tandem mass spectrometer, in particular a triple quadrupole device, in particular an automated, random-access LC-MS device.
23. The method of any of the proceeding aspects, wherein in step d) the present or level of the derivatized analyte of interest is determined by LC-MS, wherein the parent ion of derivatized valproic butylamide (N-butyl-2-propylpentanamide) is measured at a m/z value 200.2±1 in positive mode, and/or the parent ion of derivatized salicylic butylamide (2-hydroxy-N-butylbenzamide) is measured at a m/z value 191.87±1 in negative mode.

24. The method of any of the proceeding aspects, wherein step c) comprises a solvent, in particular a solvent selected from the group consisting of water, acetonitrile (CH₃CN), tetrahydrofuran (THF), Dioxanes, N,N-Dimethylformamide (DMF), Dimethyl sulfoxide (DMSO), acetone, t-butyl alcohol, diglyme, dimethyl ether (DME), methanol (MeOH), ethanol (EtOH), 1-propanol (1-PrOH), 2-propanol (2-PrOH), ethylene glycol, Hexamethylphosphoramide (HMPA), Hexamethylphosphorous triamide (HMPT), and glycerin, in particular a solvent selected from the group consisting of water, CH₃CN, THF, Dioxanes, DMF, DMSO, acetone, tert-butyl alcohol (tBuOH), diglyme, and DME.

25. The method of any of the proceeding aspects, wherein step c) comprises a non-nucleophilic base that is stable and miscible with water, in particular selected from the group consisting of 1,8-Diazabicyclo[5.4.0]undec-7-ene (DBU), trimethylamine (TEA), Diisopropylethylamine (DIPEA), Na₃PO₄, Na₂CO3, and Cs₂CO₃.

26. The method of any of the proceeding aspects, wherein in step c) the sample is treated with the nucleophilic derivatization reagent immediately after the sample is obtained, in particular within less than 10 min after the sample was obtained, in particular within less than 5 min or 3 min after the sample was obtained.
27. The method of any of the proceeding aspects, wherein in step c) the sample is treated with a nucleophilic derivatization reagent sample for more 30 seconds, in particular more than 1 min, in particular more than 2 min.
28. An analytical system adapted to perform the method of any of aspects 1 to 27.
29. The analytical system of aspect 28, which is a LC-MS device.
30. The analytical system of aspect 28 or 29, wherein the analytical system is automated.
31. The analytical system of aspect 28 or 29 or 30, which is a clinical diagnostics system.
32. A sampling tube for collecting a patient sample comprising a nucleophilic derivatization reagent for forming a derivatized analyte of interest in a sample, wherein the one or more analytes of interests is a carboxylic acid having a molar mass of smaller than 200 Da, preferably wherein the one or more analytes of interests is valproic acid or salicylic acid.
33. The sampling tube of aspect 32 comprising a device with a reservoir adapted for receiving a sample to be collected.
34. The sampling tube of aspect 32 or 33, wherein the nucleophilic derivatization reagent comprising an amine group, in particular a primary or secondary amine group, in particular a primary amine group.
35. The sampling tube of any one of aspect 32 to 34, wherein the nucleophilic derivatization reagent comprises 1 C-atom, in particular 3 to 20 C-atoms, in particular 3 to 10 C-atoms, in particular 3 to 5 C-atoms, in particular 4 C-atoms.
36. The sampling tube of any one of aspect 32 to 35, wherein the nucleophilic derivatization reagent is a linear or branched amine, in particular a linear amine, in particular a linear primary amine, in particular a linear primary amine comprising 3 to 5 C-atoms.

37. The sampling tube of any one of aspect 32 to 36, wherein the nucleophilic derivatization reagent is selected from the group consisting of propylamine, butylamine, or pentylamine, in particular primary linear butylamine.

38. The sampling tube of any one of aspect 32 to 37, wherein the nucleophilic derivatization reagent is comprised in liquid or lyophilized form.

39. The sampling tube of any one of aspect 32 to 38, wherein the nucleophilic derivatization reagent further comprises a non-nucleophilic base that is stable and miscibile with water, in particular selected from the group consisting of DBU, TEA, DIPEA, Na₃PO₄, Na₂CO₃, and Cs₂CO₃.

40. The sampling tube of any one of aspect 32 to 39, wherein the nucleophilic derivatization reagent is embodied in liquid form in a solvent, in particular a solvent selected from the group consisting of water, CH₃CN, THF, Dioxanes, DMF, DMSO, acetone, tBuOH, diglyme, DME, MeOH, EtOH, 1-PrOH, 2-PrOH, ethylene glycol, Hexamethylphosphoramide (HMPA), Hexamethylphosphorous triamide (HMPT), and glycerin, in particular a solvent selected from the group consisting of water, CH₃CN, THF, Dioxanes, DMF, DMSO, acetone, tBuOH, diglyme, and DME.

41. Use of the sampling tube of any one of aspect 32 to 40 in a method according to any of the proceeding aspects 1 to 27.

42. Use of a nucleophilic derivatization reagent in a method according to any of the proceeding aspects 1 to 27.

43. Use of the method of any of aspects 1 to 27 for determining the presence or level of one or more analytes of interest in a sample.

### Examples

The following examples are provided to illustrate, but not to limit the presently claimed invention.

### Example 1: General Method

Scheme 1 shows the a method for determining the presence or level of an analyte of interest having a molar mass of smaller than 200 Da in a sample. The analyte of interest can be e.g. valproic acid or salicylic acid. The analyte of interest is preferably a carboxylic acid. After providing the sample comprising the analyte of interest, it can be optionally activated by the addition of at least one activation reagent for forming an ester of the analyte of interest. Then, a derivatization step for forming a derivatized analyte of interest can be performed, e.g. by amidation using any primary amine, e.g. a primary alkylamine. Then, the presence or the level of the derivatized analyte of interest in the sample can be determined using mass spectrometry, e.g. in combination with a chromatographic step (e.g. LC-MS). The residues X, Y and R are independently selected from the group consisting of hydrogen, C1-C10-alkyl, C1-C10-alkenyl, C5-C10-cycloalkyl, C5-C12-aryl, C4-C10-heteroaryl and —(—O—CH₂—CH₂—)ₙ—O—CH₃ with n being an integer in the range of from 1 to 15, wherein each of X, Y may have at least one further substituent selected from the group consisting of hydrogen, C1-C5-alkyl, C5-C12-aryl, C4-C10-heteroaryl. Alternatively, X and Y are residues of an amino acid or peptide or protein, whereby

X or Y is the N- or respectively C-terminal group of the amino acid, peptide, or protein.

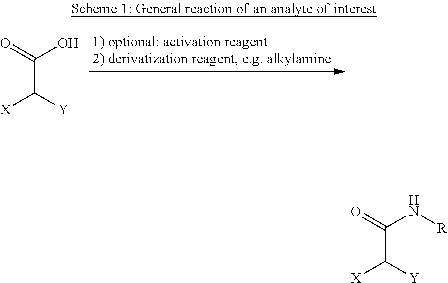

### Example 2: Valproic Acid as the Analyte of Interest

Scheme 2 shows the method for determining the presence or level of valproic acid as the analyte of interest, e.g. in serum. As an activation reagent HO-Succinimide (HO—Su) and carbodiimides (e.g. Diisopropylcarbodiimide (DIC) or N-(3-Dimethylaminopropyl)-N′-ethylcarbodiimide hydrochloride (EDC)), or a alternatives thereof, can be used. The derivatization step can be an amidation using a nucleophilic derivatization reagent, e.g. any amine, preferably primary amine. This reaction can be performed in water environment and in the presence of many other substances. Other suitable reagents for the nucleophilic derivatization reagent and/or activation reagent can be used. In the following, the inventors present data that show this reaction also works in serum using simple alkylamines like butylamine or propylamine that form valproic amides that are larger than the native compound and may therefore be properly fragmented in MS/MS. The yielding products for butylamine or propylamine as the nucleophilic derivatization reagent is numbered as 3 and 4 in Scheme 3.

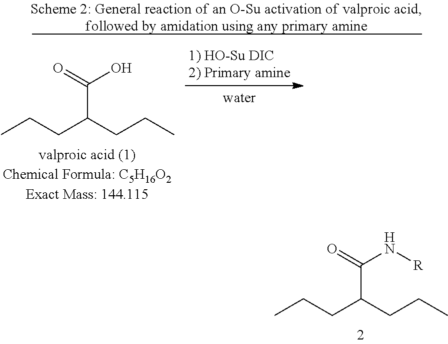

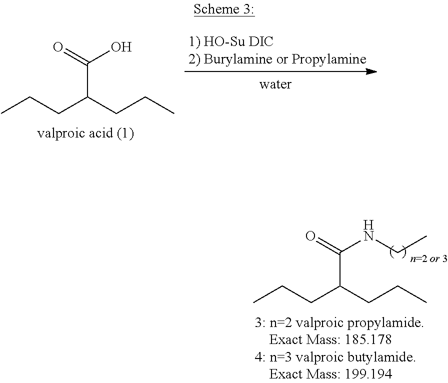

Each combination of spiked serum and serum diluted 1:10 with either butylamine or propylamine was performed in 5 replicates, giving a total of 20 samples in total. All samples were next measured using LC-MS/MS with a method that contained 3 Multiple Reaction Monitoring (MRM) transitions for valproic-butylamide and 3 more MRM transitions for valproic-propylamide. Next, all chromatograms were processed in ABSciex Multiquant software from which S/N values were obtained.

### Amidation and Purification

Valproic acid is spiked into serum to a concentration of 3 μg/mL. 50 μL of this serum was transferred into a reaction vessel and activation mixture (HOSu and DIC, 40 μL of a 50 mg/mL solution in water) was added. Following activation, either propylamine or butylamine (50 μL of a 5M solution in water) was added to allow for amidation.

Subsequently, optionally magnetic beads (bead type B) were added onto which the analytes were captured. These can then be washed twice with water (150 μL), after which the analytes were eluted using 90% MeOH in water (50 μL). The eluate (25 μL) was then transferred to a vial with micro-insert and water (25 μL) was added. This was then injected into LC-MS/MS for quantitation of the valproic amides using the method described below.

### Quantitation—δLC-Settings

Using a Poroshel-Sb-Aq column (50×2.1 mm) and water with 0.1% HCOOH as mobile phase A, and CH₃CN with 0.1% HCOOH as mobile phase B, the following gradient with flow rate 0.5 mL/min was applied (table 1):

TABLE 1

| Time (min) | A (%) | B (%) |
|---|---|---|
| 0.0 | 98 | 2 |
| 5.00 | 5 | 95 |
| 5.30 | 5 | 95 |
| 5.50 | 98 | 2 |
| 7.50 | 98 | 2 |

### Quantitation—MS/MS Settings

TABLE 2

| Q1 | Q3 | | | | | | |
|---|---|---|---|---|---|---|---|
| (precursor | (Product | Time | | | | | |
| Mass) | Mass | (msec) | ID | DP | EP | CE | CXP |
| 200.1 | 116.0 | 5 | Valp- | 10 | 10 | 27 | 8 |
| | | | butylamide- | | | | |
| | | | pos1 | | | | |
| 200.1 | 60.1 | 5 | Valp- | 10 | 10 | 27 | 8 |
| | | | butylamide- | | | | |
| | | | pos2 | | | | |
| 200.1 | 57.0 | 5 | Valp- | 10 | 10 | 45 | 8 |
| | | | butylamide- | | | | |
| | | | pos3 | | | | |
| 186.5 | 85.1 | 5 | Valp- | 1 | 10 | 25 | 22 |
| | | | propylamide- | | | | |
| | | | pos1 | | | | |
| 186.5 | 127.1 | 5 | Valp- | 1 | 10 | 21 | 14 |
| | | | propylamide- | | | | |
| | | | pos2 | | | | |
| 186.5 | 60.1 | 5 | Valp- | 1 | 10 | 27 | 24 |
| | | | propylamide- | | | | |
| | | | pos3 | | | | |
| 146.1 | 146.1 | 5 | Valproic acid- | −40 | −10 | −10 | −10 |
| | | | d3 | | | | |
| 143.1 | 143.1 | 5 | Valproic acid | −40 | −10 | −10 | −10 |

Three different MRM transitions (Q1 and Q3 mass) were monitored for valproic acid-butylamide as well as valproic acid-propylamide. Furthermore, pseudo-MRMs for valproic acid and its internal standard valproic acid-d3 were included in the MS method. Each transition was tuned individually with regard to the declustering potential (DP), entrance potential (EP), collision energy (CE) and cell exit potential (CXP).

### Chromatographic Results

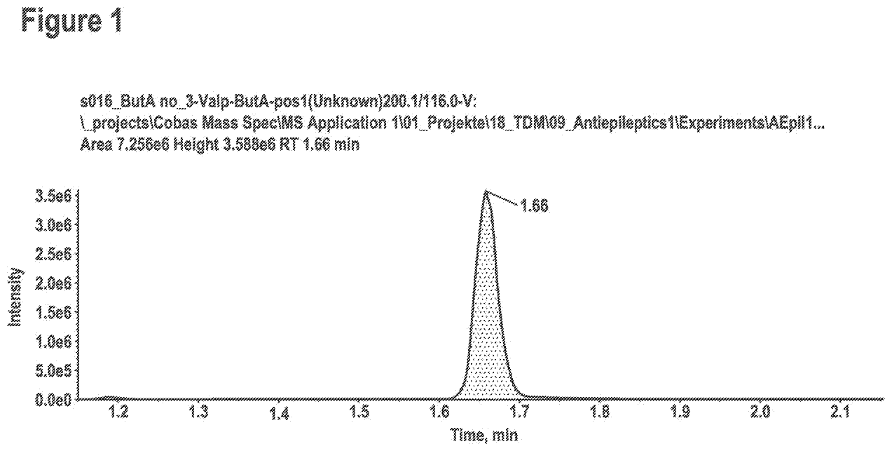

Amidation with butylamine in spiked serum to obtain product 4 and subsequent purification and LC-MS/MS as described above, a peak was obtained at 1.66 minutes with a S/N value of >9000. An example is depicted in FIG. 1 (valproic-butylamide from serum. S/N: 9152).

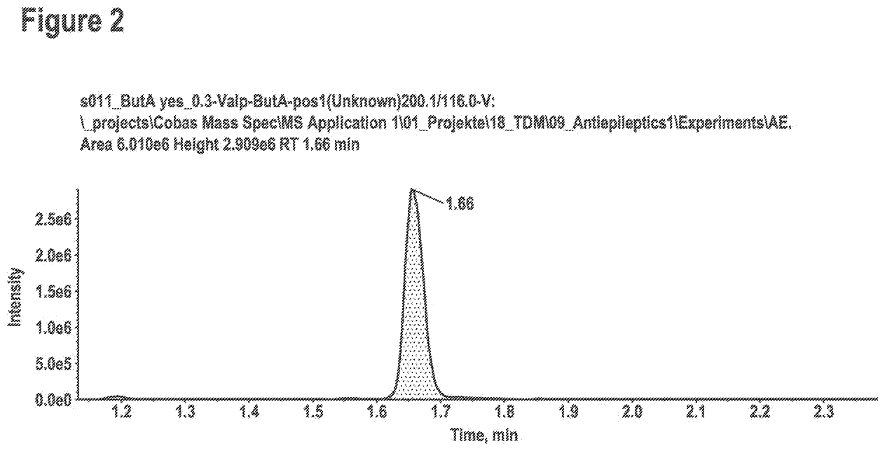

Amidation with butylamine in spiked and 1:10 diluted serum to obtain product 4 and subsequent purification and LC-MS/MS as described above, we obtain a peak at 1.66 minutes with a S/N value of >9300. An example is depicted in FIG. 2 (valproic-butylamide from serum 1:10 diluted. S/N: 9403). Given, that this product contains only 0.3 μg/ml, a S/N equal to the undiluted sample with an original concentration of 3 μg/mL is obtained. This shows that a predilution positively impacts the sensitivity.

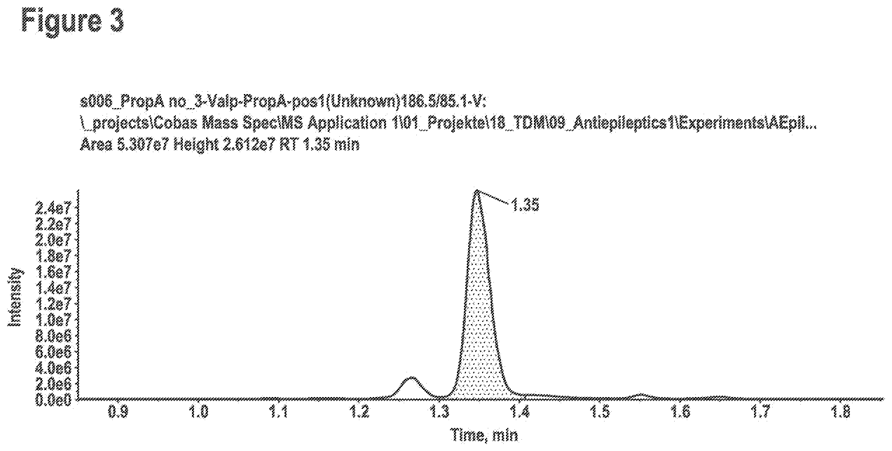

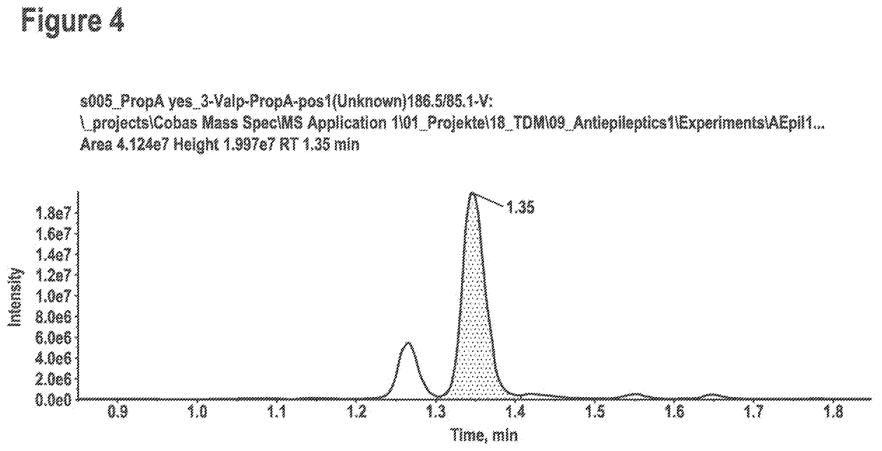

Amidation with propylamine in spiked serum to obtain product 3 and subsequent purification and LC-MS/MS as described above, we obtain a peak at 1.35 minutes with a S/N value of >12000 (FIG. 3, valproic-propylamide from serum 1:10 diluted, S/N: 12016). FIG. 4 shows, that amidation works equally well in both diluted and undiluted serum with minimal effect on S/N (FIG. 4: valproic-propylamide from serum SIN: 16991).

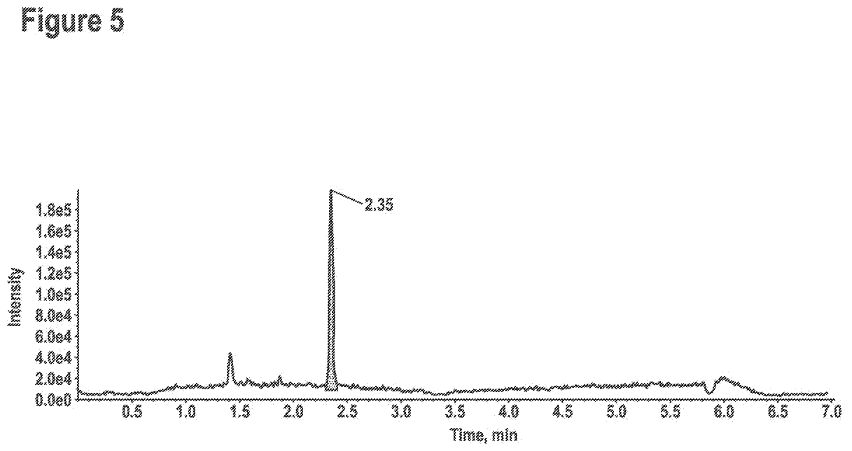

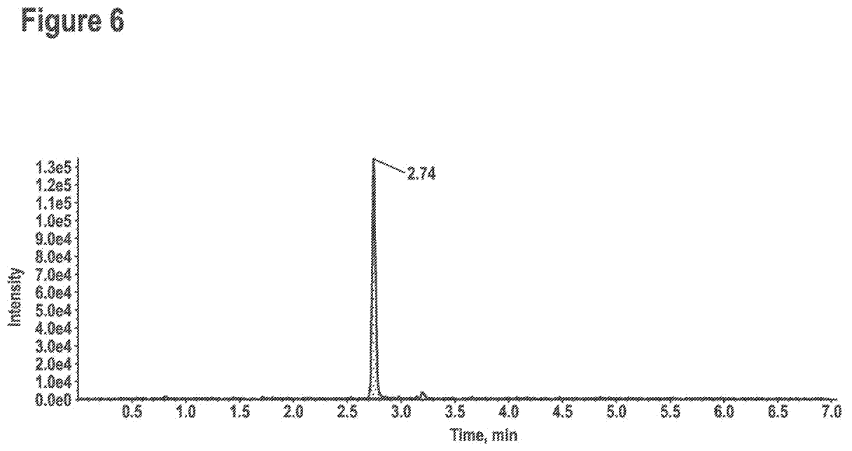

An example of two calibrators (at Limit of Quantitation (LOQ), FIG. 5 and at Upper Limit of Measuring Interval (ULMI), FIG. 6), measured via pseudo MRM, shows low S/N values, relative to the MRM transitions for the amidated products.

FIG. 5 shows Cal 3 (12 μg/mL) worked up from serum using enrichment workflow, detecting pseudo MRM 143.1/143.1. S/N: 710.5

FIG. 6. Cal 3 (12 μg/mL) worked up from serum with derivatization followed by enrichment workflow, detecting MRM 200.1/116. S/N: 2837.5

Using 3 MRM traces to quantify the obtained valporic-butylamide and 3 more for the valproic-propylamide, peak-integration shows high Signal to Noise (S/N) values that are customary for MRM transitions. Compared to current S/N values, obtained from pseudo MRM methods, the S/N values presented here are much higher.

TABLE 3
S/N values for valproic-butylamide for all 3 MRM traces.

| Predilution 1:10 | MRM trace | Signal/Noise (Mean) |
|---|---|---|
| No | Valp-butylamide-pos 1 | 8528 |
| No | Valp-butylamide-pos 2 | 50481 |
| No | Valp-butylamide-pos 3 | 8709 |
| Yes | Valp-butylamide-pos 1 | 10664 |
| Yes | Valp-butylamide-pos 2 | 55288 |
| Yes | Valp-butylamide-pos 3 | 11382 |

TABLE 4
S/N values for valproic-propylamide for all 3 MRM traces.

| Predilution 1:10 | MRM trace | Signal/Noise (Mean) |
|---|---|---|
| No | Valp-propylamide-pos 1 | 20044 |
| No | Valp-propylamide-pos 2 | 22783 |
| No | Valp-propylamide-pos 3 | 9911 |
| Yes | Valp-propylamide-pos 1 | 13896 |
| Yes | Valp-propylamide-pos 2 | 15236 |
| Yes | Valp-propylamide-pos 3 | 7442 |

The resulting products 3 and 4, that could be easily generated in an automized workflow in serum, are larger molecules that may be fragmented in the MS-analyzer, generating specific or characteristic fragments and therefore enable more sensitive and specific quantitation of valproic acid as is exemplified by the high S/N values, Residual native (i.e. unreacted valproic acid) were not observed, which shows that the reaction is kinetically very efficient.

Additionally, an isotopically labeled internal standard can be added in order to correct for any incomplete reactions.

The reaction speed and whether the reaction is completed is not of significance, since this will be the same for analyte and the internal standard. Given the high concentrations of measuring range for this compound, enough material will have reacted to allow for accurate quantitation.

The predilution of the sample is not mandatory to obtain high S/N values and high peak-intensities, especially compared to results obtained from methods with a pseudo MRM transition. Preferably, a predilution with water has a positive impact on sensitivity.

The obtained results show that using a derivatization, valproic acid can be measured using MS, preferably MRM transitions, thereby obtaining high S/N values. This allows for a more sensitive and precise quantitation of this antiepileptic drug.

### Example 3: Precision and Accuracy Comparison between Derivatization Method Versus Standard Method

Serum was spiked with valproic acid and diluted further with the same serum to obtain 6 calibration levels (see table below). Furthermore a quality control sample was prepared by spiking of a different serum sample than used for the preparation of the calibrators. The concentration of this QC sample was 11.2 μg/mL.

Next, all calibration and QC samples were processed in triplicates with a standard enrichment workflow, and with a derivatization workflow.

The standard enrichment workflow was carried out as follows: to 50 μL sample, 20 μL of a formic acid solution (100 mM) was added. To this magnetic beads (40 μL of a 50 mg/mL suspension) of bead type A were added. After incubation, the beads were washed twice with 150 μL water. Subsequently, the analyte was eluted from the beads with a 50 μL of a mixture of MeOH (90%), water (10%).

The derivatization workflow was carried out as follows: to 50 μL sample, a mixture of N-Hydroxysulfosuccinimide sodium salt and N-(3-Dimethylaminopropyl)-N′-ethylcarbodiimide hydrochloride (50 μL of a 5a mixture containing 50 mg/mL of each compound diluted in water) was added. Following incubation, butylamine (50 μL of a 5M solution in water) was added. Again following a short reaction period, magnetic beads (40 μL of a 50 mg/mL suspension) of bead type B were added. After incubation, the beads were washed twice with 150 μL water. Subsequently, the analyte was eluted from the beads with a 50 μL of a mixture of MeOH (90%), water (10%). Subsequently, this eluate (30 μL) was diluted with water (30 μL).

TABLE 5
Calibrator concentration

| Calibrator | Concentration (μg/mL) |
|---|---|
| CAL1 | 1 |
| CAL2 | 2 |
| CAL3 | 12 |
| CAL4 | 60 |
| CAL5 | 120 |
| CAL6 | 144 |

Following these sample preparations, the obtained samples were measured via LC-MS/MS. Using a Poroshel-Sb-Aq column (50×2.1 mm) and water with 0.1% HCOOH as mobile phase A, and CH₃CN with 0.1% HCOOH as mobile phase B on an Agilent Infinity II Autosampler-Pump system, the following gradient was applied (table 6):

TABLE 6
Gradient

| Time (min) | A (%) | B (%) |
|---|---|---|
| 0.0 | 98 | 2 |
| 5.00 | 5 | 95 |
| 5.30 | 5 | 95 |
| 5.50 | 98 | 2 |
| 7.50 | 98 | 2 |

For MS/MS measurements and AB-Sciex 6500+System was used with the following MS Settings (table 7) were used:

TABLE 7

| Q1 | Q3 | | | | | | |
|---|---|---|---|---|---|---|---|
| (precursor | (Product | Time | | | | | |
| Mass) | Mass | (msec) | ID | DP | EP | CE | CXP |
| 200.1 | 116.0 | 5 | Valp- | 10 | 10 | 27 | 8 |
| | | | butylamide- | | | | |
| | | | pos1 | | | | |
| 200.1 | 60.1 | 5 | Valp- | 10 | 10 | 27 | 8 |
| | | | butylamide- | | | | |
| | | | pos2 | | | | |
| 200.1 | 57.0 | 5 | Valp- | 10 | 10 | 45 | 8 |
| | | | butylamide- | | | | |
| | | | pos3 | | | | |
| 2003.1 | 119.0 | 5 | Valp-D3- | 10 | 10 | 27 | 8 |
| | | | butylamide- | | | | |
| | | | pos2 | | | | |
| 2003.1 | 63.1 | 5 | Valp-D3- | 10 | 10 | 27 | 8 |
| | | | butylamide- | | | | |
| | | | pos3 | | | | |
| 2003.1 | 60.0 | 5 | Valp-D3- | 10 | 10 | 45 | 8 |
| | | | butylamide- | | | | |
| | | | pos1 | | | | |
| 146.1 | 146.1 | 5 | Valproic | −40 | −10 | −10 | −10 |
| | | | acid-d3 | | | | |
| 143.1 | 143.1 | 5 | Valproic acid | −40 | −10 | −10 | −10 |

Following measurements, the data were processed using Multiquant version 3.03. For the data of both methods, a linear calibration was used, from which the calculated concentrations of the quality control samples were obtained. From these, the precision and accuracy was calculated.

In table 8 it can be seen that the derivatization method yields a better precision and accuracy than the method that uses a pseudo MRM. From this it can be concluded, that the here proposed method is a substantial improvement to the the state of the art.

TABLE 8

| | | Coefficient | (Accuracy) |
|---|---|---|---|
| Method | Mean | of Variance | Mean |
| Derivatization | 11.8 | 1.5 | 105.0 |
| Method | | | |
| Standard method | 11.3 | 6.0 | 90.6 |
| with pseudo MRM | | | |

This patent application claims the priority of the European patent application 20206044.8, wherein the content of this European patent application is hereby incorporated by references.

1. A method for determining the presence or level of an analyte of interest having a molar mass of smaller than 200 Da in a sample comprising the steps of
a) providing the sample comprising the analyte of interest, wherein the analyte of interest comprises a carboxylic acid group,
b) optionally activating the analyte of interest by the addition of at least one activation reagent,
c) derivatizing the analyte of interest provided by step a) or b) with a nucleophilic derivatization reagent for forming a derivatized analyte of interest, and
d) determining the presence or level of the derivatized analyte of interest in the sample using immunological assay or mass spectrometry (MS).

2. The method of claim 1, wherein the method is automated.

3. The method of claim 1, wherein the analyte of interest has a molar mass of 150 Da or less.

4. The method of claim 1, wherein the analyte of interest is valproic acid or salicylic acid.

5. The method of claim 1, wherein in step c) an amide of the derivatized analyte of interest is formed.

6. The method of claim 1, wherein the nucleophilic derivatization reagent comprises an amine group.

7. The method of claim 1, wherein the nucleophilic derivatization reagent is selected from the group consisting of propylamine, butylamine, or pentylamine, in particular primary linear butylaminc.

8. The method of claim 1, wherein the at least one activation reagent is a first activation reagent or a second activation reagent or a combination thereof,
wherein the first activation reagent is selected from the group consisting of N-Hydroxysuccinimid (OHSu), N-Hydroxysulfosuccinimide (sulfo-O Su), Hydroxybenzotriazole (HOBt) and salts of these compounds, and
wherein the second activation reagent is selected from the group consisting of Dicyclohexylcarbodiimid (DIC), 1-Ethyl-3-(3-dimethylaminopropyl)carbodiimid (EDC), N-(3 -Dimethylaminopropyl)-N′-ethylcarbodiimide, N,N′-Dicyclohexylcarbodiimide, N-Cyclohexyl-N′-(2-morpholinoethyl)carbodiimide methyl-p-toluenesulfonate, 1,3-Bis(trimethylsilyl)carbodiimide and N,N′-Methanetetraylbis[4-methyl]benzenamine.

9. The method of claim 1, wherein the analyte of interest is free of a nucleophilic functional group, which could react with the carboxylic acid group of the analyte of interest.

10. The method of claim 1, wherein the said method comprises an additional step:
e) enriching the sample.

11. An analytical system adapted to perform the method of claim 1.

12. A sampling tube for collecting a patient sample comprising a nucleophilic derivatization reagent for forming a derivatized analyte of interest in a sample, wherein the one or more analytes of interest is a carboxylic acid having a molar mass of smaller than 200 Da.

13. (canceled).

14. (canceled).

15. (canceled).

16. The method of claim 6, wherein the nucleophilic derivatization reagent comprises a primary amine group, a secondary amine group, or a tertiary amine group.

17. The method of claim 16, wherein the nucleophilic derivatization reagent comprises a secondary amine group.

18. The method of claim 16, wherein the nucleophilic derivatization reagent comprises a primary amine group.

19. The method of claim 7, wherein the nucleophilic derivatization reagent comprises primary linear butylamine.

20. The method of claim 10, wherein in step e) the sample is enriched using magnetic beads.

21. The sampling tube of claim 12, wherein the one or more analytes of interest is valproic acid or salicylic acid.
